(19) United States
(12) Patent Application Publication
(10) Pub. No.: US 2010/0222862 A1
(43) Pub. Date: Sep. 2, 2010

(54) Stent, Stent Graft and Other Implantable Assemblies

(75) Inventor: Palle M. Hansen, Bjaeverskov (DK)

Correspondence Address: BRINKS HOFER GILSON & LIONE/CHICAGO/COOK, PO BOX 10395, CHICAGO, IL, 60610, US

(73) Assignees: Cook Incorporated, Bloomington, IN (US); William Cook Europe ApS, Bjaeverskov (DK)

(21) Appl. No.: 12/676,548

(22) PCT Filed: Sep. 5, 2008
(86) PCT No.: PCT/US08/75450

§ 371 Date: Apr. 28, 2010

Related U.S. Application Data
(60) Provisional application No. 60/967,628, filed on Sep. 6, 2007.

(51) Int. Cl. A61F 2/84; A61F 2/82; A61F 2/01

(52) U.S. Cl. 623/1.11; 623/1.13; 623/1.15; 606/200

(57) ABSTRACT

A stent or other implantable medical device is provided with one or more gripping shoulders which are able, when the stent is compressed onto a delivery cannula to frictionally mechanically engage the outer surface of the catheter so as to grip thereon and maintatin the position of the stent relative to the cannula during withdrawal of the covering sheath.

### FIELD OF THE INVENTION

The present invention relates to a stent, a stent graft and other implantable medical devices and to an introducer or deployment assembly for deploying implants and other prostheses within a patient.

### BACKGROUND OF THE INVENTION

A typical endoluminal introducer or deployment system includes a device carrier usually in the form of an inner catheter or cannula, which may also be arranged as a pusher and/or dilator (hereinafter referred to as an inner catheter or catheter element). A sheath covers the inner catheter. An implant or prosthesis is carried on the inner catheter and is fixed thereto by means of the covering sheath and with or without one or more restraining wires or any of a number of other known retention systems.

The implant or prosthesis might be a stent, a stent graft, a filter, an occlusion device or any other implantable device of such a nature.

Once the distal end of the catheter has been positioned inside a patient, typically at the site of the patient's vasculature to be treated, the device is released and deployed in the desired position. The deployment operation involves retracting the covering sheath so as to expose the device to be implanted, which device is then deployed, either by self-expansion or by means of an expansion device such as an inflatable balloon. In the case where the device is also held by restraining wires, these are withdrawn typically after retraction of the sheath. Restraining wires may or may not be used in such apparatus, generally in dependence upon the nature of the device to be deployed, size restrictions and the particular medical application or intervention procedure.

The step of retracting the covering sheath from the inner catheter has been known to compress or otherwise deform the device to be implanted, particularly when restraining wires are not used. This can affect the positioning of the device at the deployment site and in some circumstances can damage the device itself. These problems can be experienced particularly in the case of delicate implants such as some stents.

Various systems have been proposed to deal with this problem. US Patent Publication No. 2004/0106977 discloses in some embodiments the provision of one or more bands of an adhesive on the outer surface of the inner catheter, which are intended to hold a stent until its deployment. In other embodiments ridges or stepped walls on the outer surface of the inner catheter engage struts of the stent to prevent longitudinal movement thereof along the inner catheter as the covering sheath is retracted.

A problem with providing adhesive on the inner catheter is that this is another material to which a patient is exposed, even if only temporarily. It also requires a constant compressive force on the device held on the inner catheter for the glue to perform its function fully. The pressure required to compress the stent reliably into the adhesive layer results in there being a higher friction between the sheath and the stent, which provides an undesirable compromise in such devices.

The mechanical holding function provided by ridges or stepped walls on the inner catheter can be significantly better at holding the device firmly on the inner catheter during the deployment operation. However, there are risks that the ridges on the outer surface of the inner catheter can snag on the device once this has been deployed and can grate against the inner surfaces of the patient's vasculature as it is retracted from within the patient. This can cause movement or damage to the implanted device and irritation or damage to the patient's vasculature or organs. The risks are increased where the device to be implanted is small and/or particularly delicate and when the device is implanted in or near a tortuous part of a patient's vasculature.

U.S. Pat. No. 6,979,346 discloses a mechanism having the purpose of retaining the stent on the delivery balloon. The internal walls of the stent are roughened so as to increase friction between the stent and the delivery balloon. The roughening is in the form of asperities or grooves along the inside of the stent or part of the stent. It is described that the roughened areas can usefully be coated with a bio-compatible layer so as to reduce interaction between the roughened area(s) of the stent and any blood flowing through the body lumen where the stent is implanted.

U.S. Pat. No. 6,240,978 discloses a stent structure provided with inwardly or outwardly formed elevations which reduce the friction between the stent and the shaft of the positioning instrument/catheter.

US Patent Publication No. 2006/0004436 discloses a stent structure having arcuate struts. It is considered that the struts flatten on compression of the stent and acquire their concave shape when the stent is expanded.

US Patent Publication No. 2004/0236405 discloses a stent with variable wall thickness for the specific purpose of increasing its flexibility along its length. In one example the ends of the stent itself are made of thicker material, while the central portion of the stent is made of thinner material. One embodiment describes a textured surface on the outside of the stent.

### SUMMARY OF THE INVENTION

The present invention seeks to provide an improved deployment assembly and an improved stent, stent graft or other implantable medical device.

According to an aspect of the present invention, there is provided an implantable medical device which is compressible onto a delivery device, the implantable medical device including at least one internal wall providing a bore within the device, said at least one internal wall including a substantially entirely smooth internal surface and at least one gripping shoulder able to extend internally into the bore.

The gripping shoulder or shoulders may be an edge of the internal wall of the medical device and/or a sharp protrusion. In the preferred embodiment, the gripping shoulders are of a type able to grip onto a carrier element, such as a catheter, cannula or pusher member, by indenting the outer wall of such a carrier.

In one embodiment, the or each internal wall is provided with one or more sharp protrusions extending from a smooth inner wall surface. This has the advantage that the internal wall or walls of the device do not provide asperities or grooves which can interact with blood components.

Advantageously, the gripping member or members are designed to extend into the bore when the medical device is radially compressed. In this embodiment, the internal wall or walls preferably provide a substantially cylindrical or smooth internal surface throughout the length of the bore. Thus, when the device is in its deployed state, the internal surface or surfaces of the device present no additional surface for trapping blood components over and above existing devices.

In one embodiment, the device is or includes a stent, wherein one or more of the stent struts is provided with a sharp protrusion or indentation extending internally therewithin.

In another embodiment, the device is provided with a plurality of internal walls, wherein each includes means for providing an internally extendable shoulder.

In one embodiment, the device includes a plurality of stent sections, wherein each section has a varying thickness in a longitudinal direction of the device. Preferably, facing ends of each section have differing thicknesses. Advantageously, each stent section has a wedge shape in longitudinal cross-section. It is preferred that the stent sections have an uncompressed configuration in which the internal walls of the stent section provide a substantially smooth internal surface to the device.

The varying thicknesses of the sections of the device cause the stent sections to present internally extending shoulders when the device is compressed radially.

In another embodiment, there is provided at least one externally extending protrusion on of the device, which protrusion is operable to push an edge of the device into the bore thereof when the device is radially compressed. Advantageously, the device is provided with a plurality of internal walls and a plurality of externally extending protrusions, one for each wall.

In this embodiment, the device can have a substantially conventional form and thus can exhibit properties which are in all material respects the same as known devices, with the additional advantage of incorporating a mechanism to hold the device on a delivery catheter or cannula. Furthermore, in the embodiments which provide an externally extending biasing member, such as a thicker stent wall section or protrusion, these parts of the device can assist in fixing the device to the internal walls of a patient's vasculature by providing features which press into the vasculature. In some instances this can replace barbs and other such anchoring members.

Preferably, the internal wall or walls have surface imperfections of less than 100 nm. In the preferred embodiments, the internal walls have the same characteristics as existing devices, such as existing stents.

Advantageously, the device has an expanded shape in which the internal wall or walls provide a substantially cylindrical inner surface, that is with no noticeable inwardly extending protrusions. In this embodiment, the gripping shoulders extend into the bore of the device when the device is compressed.

The implantable medical device may be a stent, a stent graft, a vena cava filter, an occlusion device or any other similar device.

According to another aspect of the present invention, there is provided an introducer or delivery assembly including a carrier element; an implantable medical device as specified herein and an element for compressing the implantable medical device onto the carrier.

The carrier element may be a catheter, cannula, pusher member or other known carrier.

Advantageously, the compression element is a sheath.

According to another aspect of the present invention, there is provide a method of assembling an implantable medical device onto a delivery device, including the steps of locating the implantable medical device on the delivery device such that a carrier element of the delivery device sits within a bore of the medical device, compressing the implantable medical device onto the carrier element such that one or more shoulders or protrusions of the implantable medical device extend internally into the bore and onto the carrier device.

Preferably, the method includes the step of radially compressing the implantable medical device such that the one or more shoulders or protrusions indent the outer wall of the carrier element.

### DESCRIPTION OF THE DRAWINGS

Embodiments of the present invention are described below, by way of example only, with reference to the accompanying drawings, in which:

FIGS. 1 and 2 are perspective views of an example of known stent delivery device which can be modified to include a catheter element according to the teachings herein;

FIG. 3 is a schematic view in cross-section of an embodiment of stent;

FIG. 4 is a schematic view in cross-section of the stent of FIG. 3 compressed onto a delivery catheter;

FIG. 5 is a schematic view in cross-section of another embodiment of stent;

FIG. 6 is a schematic view of an embodiment of mandrel and protrusion forming assembly for use in the manufacture of the stent of FIGS. 3 and 4;

FIG. 7 is a schematic view in cross-section of another embodiment of stent;

FIG. 8 is a schematic view in cross-section of the stent of FIG. 7 compressed onto a delivery catheter;

FIG. 9 is a schematic view in cross-section of another embodiment of stent; and

FIG. 10 is a schematic diagram showing the stent of FIG. 9 compressed by a delivery sheath.

### DESCRIPTION OF THE PREFERRED EMBODIMENTS

It is to be understood that the Figures are schematic and do not show the various components in their actual scale. In many instances, the Figures show scaled up components to assist the reader.

In this description, when referring to a deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment of treatment apparatus.

On the other hand, when referring to an implant such as a stent or stent graft, the term proximal refers to a location which in use is closest to the patient's heart, in the case of a vascular implant, and the term distal refers to a location furthest from the patient's heart.

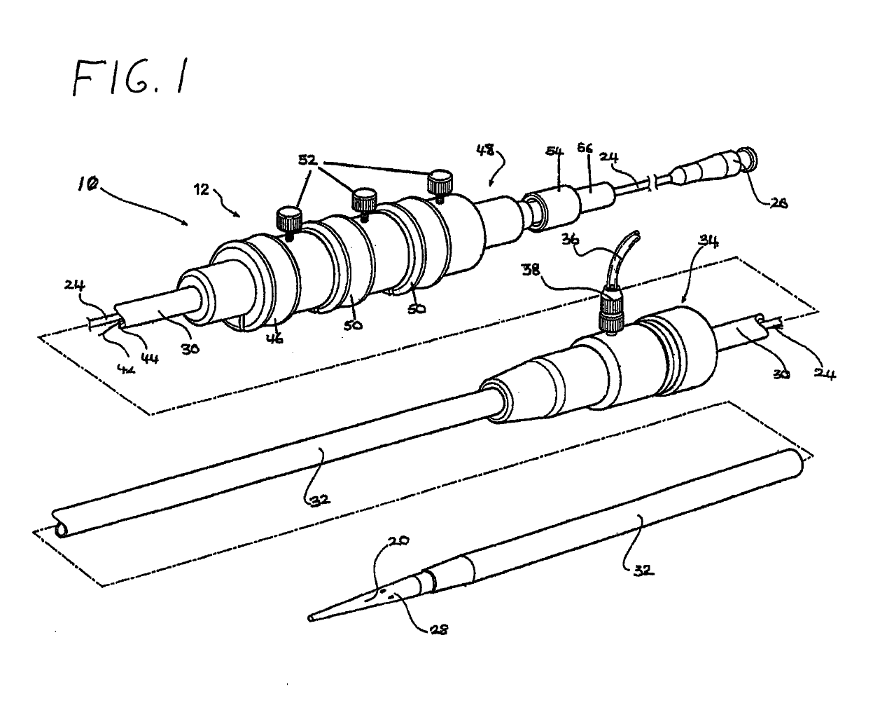

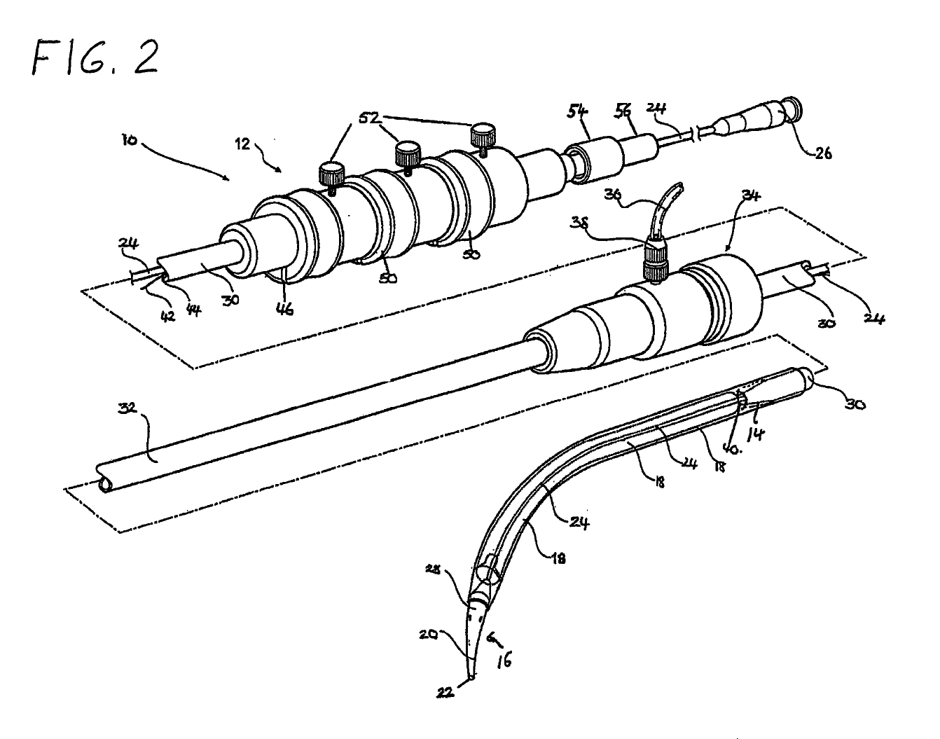

Referring to FIGS. 1 and 2, the introducer 10 includes an external manipulation section 12, a proximal attachment region 14 and a distal attachment region 16. The proximal attachment region 14 and the distal attachment region 16 secure the two ends of the implant 18. During the medical procedure to deploy the implant 18, the proximal and distal attachment regions 14 and 16 will travel through the patient's vasculature, in this example, to a desired deployment site. The external manipulation section 12 at the proximal end of the assembly 10, which is operated by a surgeon to manipulate the introducer, remains outside of the patient throughout the procedure.

The distal attachment region 16 of the introducer 10 includes a dilator tip 20, which is typically provided with a bore 22 therein for receiving a guide wire (not shown) of conventional type. The longitudinal bore 22 also provides a channel for the introduction of medical reagents. For example, it may be desirable to supply a contrast agent to allow angiography to be performed during placement and deployment phases of the medical procedure.

An inner catheter or cannula 24, conventionally made from a flexible thin walled metal tube, is fastened to the dilator tip 20. The inner catheter 24 is flexible so that the introducer 10 can be advanced along a relatively tortuous vessel, such as a femoral artery, and so that the distal end of the assembly 10 can be longitudinally and rotationally manipulated. The inner catheter 24 carries a stent 18 or other device to be implanted in the patient. The catheter 24 extends through the introducer 10 to the manipulation section 12, terminating at a connection device 26, in conventional manner.

The connection device 26 is designed to accept a syringe to facilitate the introduction of reagents into the inner catheter 24 and for this purpose is typically provided with a threaded luer lock connection.

Where provided, a pusher sheath or rod 30 (hereinafter referred to as a pusher member), typically made from a plastics material, is mounted coaxial with and radially outside of the inner catheter 24. The pusher member 30 is “thick walled”, that is the thickness of its wall is preferably several times greater than that of the guide wire catheter 24. In some instances, the pusher member 30 and the inner catheter 24 are the same component, possibly having different outer diameters at the location at which the stent 18 is to be carried.

A sheath 32 extends coaxially over and radially outside of the pusher member 30. The pusher member 30 and the sheath 32 extend distally to the manipulation region 12.

The implant 18, which may be a stent, a stent-graft or any other implant or prosthesis deliverable by this device 10, is retained in a compressed condition by the sheath 32. The sheath 32 extends proximally to a sheath manipulator and haemostatic sealing unit 34 of the external manipulation section 12. The haemostatic sealing unit 34 includes a haemostatic seal (not shown) and a side tube 36 held to the unit 34 by a conventional luer lock 38.

The sheath manipulator and haemostatic sealing unit 34 also includes a clamping collar (not shown) that clamps the sheath 32 to the haemostatic seal and a silicone seal ring (not shown) that forms a haemostatic seal around the pusher rod 30. The side tube 38 facilitates the introduction of medical fluids between the pusher rod 30 and the sheath 32. Saline solution is typically used.

During assembly of the introducer 10, the sheath 32 is advanced over the proximal end of the dilator tip 20 of the proximal attachment region 16 while the implant 18 is held in a compressed state by an external force. A suitable distal attachment (retention) section (not visible in this view) is coupled to the pusher rod 30 and retains a distal end 40 of the prosthesis 18 during the procedure. The distal end of the prosthesis 18 may be provided with a loop of material (not shown) through which a distal trigger wire 42 extends. The distal wire also extends through an aperture (not shown in FIGS. 1 and 2) in the proximal attachment section 40 into an annular region 44 between the inner catheter 24 and the pusher rod 30. The distal trigger wire 42 extends through the annular space 44 to the manipulation region 12 and exits the annular space 44 at a distal wire release mechanism 46.

A proximal portion of the external manipulation section 12 includes at least one release wire actuation section 50 mounted on a body 48, in turn mounted onto the pusher member 30. The inner catheter 24 passes through the body 48. The distal wire release mechanism 46 and the proximal wire release mechanism 50 are mounted for slidable movement on the body 48.

The positioning of the proximal and distal wire release mechanisms 46 and 50 is such that the proximal wire release mechanism or mechanisms 50 must be moved before the distal wire release mechanism 46 can be moved, such that the proximal end of the implant, that is the end of the implant which will be upstream in the direction of fluid flow in the patient's vasculature, is released first. Therefore, the distal end of the implant 18 cannot be released until a self-expanding zigzag stent thereof has been released. Clamping screws 52 prevent inadvertent early release of the prosthesis 18. A haemostatic seal (not shown) is included so that the release wires can extend out through the body 48 without unnecessary blood loss during the medical procedure.

A proximal portion of the external manipulation section 12 includes a pin vice 54 mounted onto the proximal end of the body 48. The pin vice 54 has a screw cap 56. When screwed in, vice jaws (not shown) of the pin vice 54 clamp against or engage the guide wire catheter 24. When the vice jaws are engaged, the inner catheter 24 can only move with the body 48 and hence it can only move with the pusher member 30. With the screw cap 56 tightened, the entire assembly can be moved together as one piece.

Once the introducer assembly 12 is in the desired deployment position, the sheath 32 is withdrawn to just proximal of the distal attachment section 14. This action releases the middle portion of the implant 18, in this example a stent or stent-graft, so that it can expand radially. Consequently, the stent or stent-graft 18 can still be rotated or lengthened or shortened for accurate positioning. The proximal end self-expanding stent however, is still retained at the dilator tip 16 by means of the release wires. Also, the distal end of the stent or stent-graft 18 will still retained within the sheath 32.

Next, the pin vice 54 is released to allow small movements of the inner catheter 24 with respect to the pusher rod 30 to allow the stent or stent-graft 18 to be lengthened, shortened, rotated or compressed for accurate placement in the desired location within the lumen. X-ray opaque markers (not shown) may be placed along the stent or stent-graft 18 to assist with placement of the prosthesis.

When the proximal end of the stent or stent-graft 18 is in place, the proximal trigger wire (not shown) is withdrawn by movement of the proximal wire release mechanism. The proximal wire release mechanism 50 and the proximal trigger wire can be completely removed by passing the proximal wire release mechanism 50 over the pin vice 54, the screw cap 56 and the connection unit 26.

Next, the screw cap 56 of the pin vice 54 is loosened, after which the inner catheter 24 can be pushed in a distal direction, that is towards the inside of the patient, so as to move the dilator tip 20 in a distal direction. This fully releases the proximal end of the stent or stent-graft 18, allowing it to expand so as to engage the lumen walls of the artery or vein. From this stage on, the proximal end of the stent or stent-graft 18 cannot be moved again.

Once the proximal end of the stent or stent-graft 18 is anchored, the sheath 32 is withdrawn distally of the proximal attachment section 14, which withdrawal allows the distal end of the stent or stent-graft 18 to expand. Until this point and in particular until the distal release mechanism 46 is actuated to release the distal trigger wires from the distal end of the stent 18, the distal may still be repositioned as needed.

A problem can occur during the deployment of the stent 18, in particular as the sheath 32 is retracted along the inner catheter 24. This can be particularly acute in cases where the stent is of a very flexible nature, such as a dissection stent. Friction between the inner wall of the sheath 32 and the stent 18 can cause the stent 18 to deform as the sheath is retracted, which can either adversely affect the positioning of the stent or in the worst case can result in an abortive procedure.

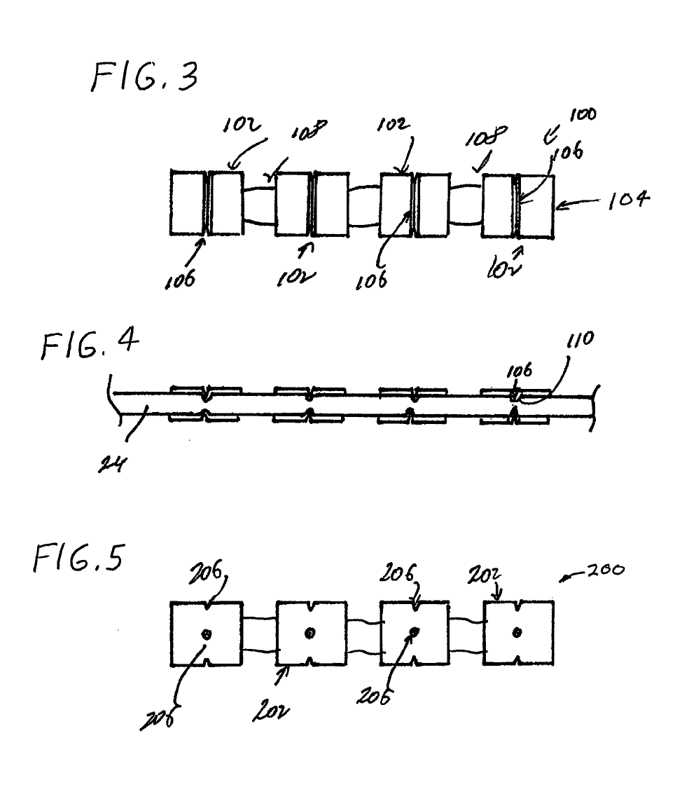

FIGS. 3 to 9 show various embodiments of stent structure which can mitigate or eliminate such disadvantages.

The embodiments of FIGS. 3 to 5 show stent structures very much in schematic form. It will be appreciated that these embodiments are in practice directed to modifications of conventional stent structures used in the art including, for example, the applicant's Zilver™ and Gianturco™ stents.

Referring to FIG. 3, this shows in schematic form a side elevational cross-sectional view of an embodiment of stent 100. This stent 100 is provided, in this example, with a plurality of annular stent sections 102 each formed of an interconnected annular series of stent struts. A lumen or bore 104 extends through the middle of the stent 100. The stent sections 102 are typically connected to one another by suitable connecting members 108, conventionally tie bars. In this regard, the stent 100 is similar to existing stents including, for example, the Zilver™ and Gianturco™ stents available from the applicant. In other words, each stent section 102 may be a ring of interconnected stents arranged in zig-zag fashion.

In some medical applications, the stent sections 102 may be separate from one another.

In this embodiment, in each stent section there is provided a gripping shoulder formed by an internally extending sharp annular rib or tooth 106. As explained in further detail below, in one embodiment, the rib 106 is created by bending the struts of the stent section 102 inwardly after formation of the stent 100. In another embodiment, there may be welded or otherwise affixed to the internal surface of the stent sections 102 a sharp annular rib of a similar structure to the rib 106 shown in FIG. 3. It will be appreciated that in the case of a stent formed by a series of interconnected stent struts, the ribs 106 will not be continuous but will be indentations of individual stent struts. Nevertheless, it is envisaged in this embodiment that when looked at as a whole the stent struts present a series of ribs which are annular in their arrangement.

FIG. 3 shows very much in schematic form each stent section 102 being provided with an internally extending rib 106. However, this may not be necessary in all applications. In some instances, for example, it may be necessary or desirable to have such ribs 106 only in some of the stent sections 102, for example in the two end sections.

Referring now to FIG. 4, the stent 100 of FIG. 3 is shown compressed onto a carrier catheter such as the inner catheter 24 of the assembly of FIGS. 1 and 2. Typically, it is the sheath 32 which compresses the stent 100 in this manner.

As can be seen in FIG. 4, as a result of the relative softness of the inner catheter 24, the annular ribs 106 deform the surface of the inner catheter 24 to form, preferably, temporary indentations 110. It will be appreciated that the depth of the indentations 110 need only be very slight and in some cases hardly noticeable to the naked eye, as long as this is sufficient to provide a good hold of the stent 102 in the longitudinal direction of the catheter 24. Whether or not indentations are formed is dependent upon the nature and material of the inner catheter 24 and the extent to which the medical device is compressed onto it. In some cases there may be no noticeable indentations at all, only a pressing of the points onto the surface of the inner catheter in order to increase friction. In other words, the annular ribs 106 are configured to engage the surface of the inner catheter 24, either mechanically and/or frictionally, in a manner sufficient to prevent movement there between during retraction of the sheath (i.e., during deployment of the stent 100).

In this state, the stent 100 is held securely on the catheter 24 even during withdrawal of the outer sheath 32. Once the sheath is withdrawn, the stent expands in conventional manner, either by self-expansion or by an expansion device such as a balloon.

FIG. 5 shows another embodiment of stent 200, similar to the stent 100 but having in place of an annular rib 106 a plurality of discrete projecting points 206. The points 206 could be formed in a similar manner as the ribs 106, that is by indentation of only a proportion of the stent struts forming one of the annular stent segments 102. In FIG. 5, only four indentations 206 are provided around each stent section 206. Any other number could be provided, including two, three and even more than four. This will primarily be dependant upon the nature of the stent, the delivery assembly and designer preference.

Referring now to FIG. 6, there is shown in cross-section one embodiment of a system for producing a stent of the type shown in FIGS. 3 and 4. The system includes a generally cylindrical mandrel 150 having a pointed tip 152. Along the length of the barrel 154 of the mandrel 150 there are provided a plurality of spaced annular recesses or grooves 156 extending radially around the outer surface of the mandrel 150.

Aligned with the recesses 156 are a plurality of pointed deforming elements 158. The elements 158 are able to move towards and away from the recesses 156 and in some embodiments into the recesses themselves.

In order to form a stent 100 having the characteristics of FIG. 1, once the tubular stent has been formed, for example of a Zilver or Gianturco stent manufactured by the applicant, it is fitted onto the mandrel by sliding it from the pointed end 152. For this purpose, the mandrel 150 typically has an outer diameter at least as large as the inner diameter of the stent 100. During this operation, the deforming elements 158 are in a retracted position.

Once the stent 100 has been located over the mandrel 150 with the stent sections 102, and in particular the struts thereof, overlying the recesses 156, the deforming elements 158 are moved onto the mandrel 150 to push a part of the stent struts into the recesses, thus forming the internally projecting ribs 106 shown in FIGS. 3 and 4. Once so formed, the deforming elements 158 are moved away from the mandrel and the stent 100 can then be slipped off. For this purpose, it is preferred that the recesses 156 have sloping walls to assist in the withdrawal of the stent ribs 106 therefrom.

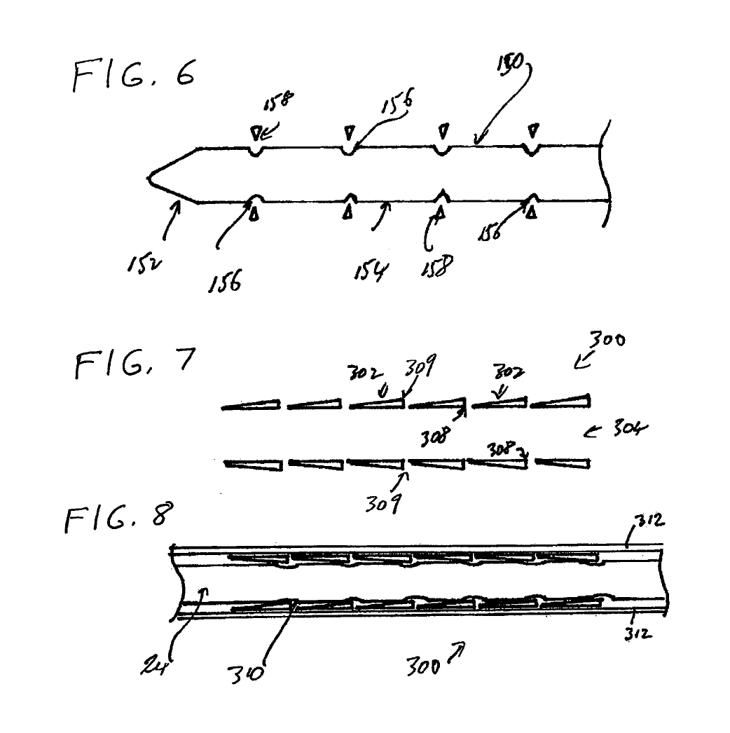

FIGS. 7 and 8 show in schematic form another embodiment of stent 300. In this embodiment, the stent 300 is formed of a plurality of interconnected stent sections or rings 302 (the interconnections not being shown for the sake of simplicity), with each section being formed by an arrangement of connected stent struts connected together in zag-zag fashion into a ring in conventional manner. This embodiment differs from known stents in that the struts forming the stent sections 302 have varying thicknesses, in this example being of a wedge shape when viewed in longitudinal cross-section as shown in FIGS. 7 and 8. They are constructed such that each stent ring or section 302 has a thicker end and a thinner end, thereby being wedge shaped when viewed in longitudinal cross-section.

It is preferred that the stent is formed so that the internal bore 304 of the stent 300 presents a substantially smooth internal surface with no projections therein, in this example being substantially cylindrical along its entire length.

Referring now to FIG. 8, when the stent 300 is compressed onto the delivery catheter 24, typically by the outer sheath 312, the sheath 312 presses against the outermost edges 309 of the stent sections 302 to cause these to deflect or bend inwardly. In effect, this is achieved as a result of the outer surfaces of the stent 300 taking the shape of the internal surface of the sheath 312, which may comprises a reinforced sheath wall to prevent the deformation thereof.

As can be seen in FIG. 8, when so compressed, the inner edges 308 of the thicker ends of the stent sections 302 are pushed inwardly so as to project into the bore of the stent 300. They thus dig into the outer surface of the catheter 24 by indenting it at locations 310. The edges 308 thus provide shoulders which in practice fix the stent 300 in the longitudinal direction of the catheter 24 until the compressive pressure of the sheath 312 is removed, at which point the stent 300 is free to expand and thus to clear the catheter 24 in the normal manner. In particular, the sheath 312 is withdrawn in a proximal direction, which is towards the right of the drawing sheet in the illustrated embodiment, so that sheath 312 slides over the outer surface of the stent 300.

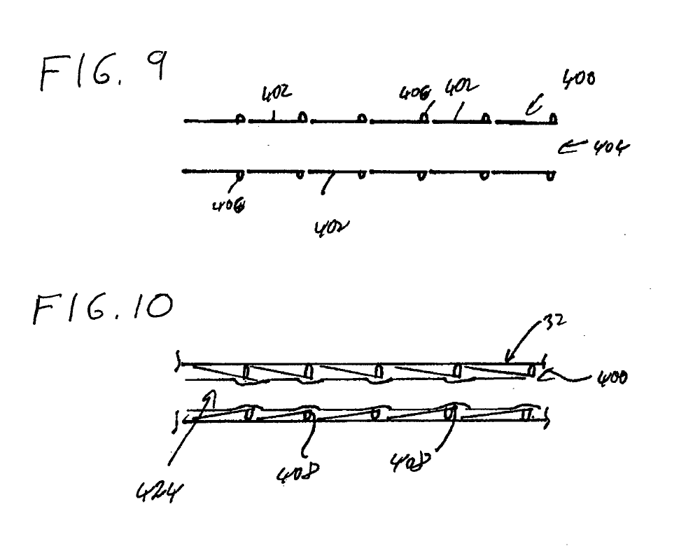

FIG. 9 shows another embodiment of stent 400 formed of standard stent sections 402 to which there is formed at one end of each of which an outwardly extending protrusion or annular series of protrusions or rivet heads 406. The stent 400 has a smooth internal bore 404.

The protrusions 406 can be formed by welding or kneading a suitable metallic or metal alloy element to the outside of the stent struts forming the stent section 402 or by deformation of the stent struts, in a manner analogous to the embodiment of FIGS. 3 and 4.

As can be seen in FIG. 10, when the stent 400 is compressed by a delivery sheath 32, the protrusions 406 push their end of the stent struts forming the stent sections 402 inwardly such that these ends present an internally extending shoulder 408 able to dig into the inner catheter 424 on which the stent 400 is carried.

An alternative to the embodiment of FIG. 10 provides the protrusions or rivet heads 406 on the internal surfaces of the stent struts 402.

In all of the described embodiments, there is provided a mechanism for holding the stent 100, 200, 300, 400 on the delivery catheter is such a manner that any deformation or movement of the stent due to withdrawal of the outer sheath 32 is substantially prevented. This is achieved without having to provide on the delivery catheter protruding bosses or edges and without the use of adhesives.

It will be appreciated that the specific embodiments show a stent which is substantially cylindrical. However, this is not to be construed as the only form of stent as the teachings herein could be applied to other shapes of medical device including devices having a conical internal bore. It will also be appreciated that the internal surfaces of the stent struts will be comparable with existing stents, that is will be substantially smooth. Typical surface roughness will be less than 100 nm, preferably less than 50 nm, more preferably less than 25 nm and most preferably less than 10 nm.

Furthermore, although the specific embodiments which have been described relate to stents, the teachings herein can be applied equally to other medical devices including and not limited to stent grafts, filters and occlusions devices.

It will be appreciated that the various features of the protrusions and shoulders disclosed herein may be combined with one another as desired by the skilled person and are not restricted to the particular embodiments in which they are described.

1. An implantable medical device which is compressible onto a delivery element, the implantable medical device including at least one internal wall providing a bore within the device, said at least one internal wall including a substantially smooth internal surface and at least one gripping shoulder able to extend internally into the bore.

2. The implantable medical device according to claim 1, wherein the at least one gripping shoulder comprises at least one of an edge of the internal wall of the medical device and a sharp protrusion.

3. The implantable medical device according to claim 1, wherein the gripping shoulder is configured to grip onto a carrier element of a delivery element by frictionally engaging or indenting the outer wall of such carrier element.

4. The implantable medical device, according to claim 1, wherein the internal wall is provided with one or more sharp protrusions extending from a substantially smooth inner wall surface, each protrusion providing a gripping shoulder.

5. The implantable medical device according to claim 1, wherein the gripping shoulder or shoulders are designed to extend into the bore when the medical device is radially compressed.

6. The implantable medical device according to claim 1, wherein the internal wall provides a substantially uniform internal surface substantially throughout the length of the bore when the device is in an expanded condition.

7. The implantable medical device according to claim 1, wherein the device comprises a stent.

8. The implantable medical device according to claim 7, wherein the stent comprises one or more stent struts provided with a sharp protrusion or indentation extending internally within the stent.

9. The implantable medical device according to claim 1, wherein the device is provided with a plurality of internal walls, wherein each wall includes means for providing an internally extendable gripping shoulder.

10. The implantable medical device according to claim 1, wherein the device comprises a plurality of stent sections, wherein each stent section has a varying thickness in a longitudinal direction of the device.

11. The implantable medical device according to claim 10, wherein facing ends of each adjacent stent section have differing thicknesses.

12. The implantable medical device according to claim 11, wherein each stent section has a wedge shape in longitudinal cross-section.

13. The implantable medical device according to claim 10, wherein the plurality of stent sections have an uncompressed configuration in which the internal walls of the stent section provide a substantially uniform internal surface to the device.

14. The implantable medical device according to claim 1, wherein there is provided at least one externally extending protrusion on an outer surface of the device, which protrusion is operable to push an edge of the device into the bore when the device is radially compressed.

15. The implantable medical device according to claim 14, wherein the device is provided with a plurality of internal walls and a plurality of externally extending protrusions.

16. The implantable medical device according to claim 1, wherein the internal wall or walls have surface imperfections of less than 100 nm.

17. The implantable medical device according to claim 1, wherein the implantable medical device is a stent, a stent graft, a filter or an occlusion device.

18. delivery assembly including a carrier element; an implantable medical device according to any preceding claim and an element for compressing the implantable medical device onto a carrier element of the delivery assembly.

19. The delivery assembly according to claim 18, wherein the carrier element is a catheter, cannula or pusher member.

20. The delivery assembly according to claim 18, wherein the compressing element is a sheath.

21. A method of assembling an implantable medical device onto a delivery device, including the steps of locating the implantable medical device on the delivery device such that a carrier element of the delivery device sits within a bore of the medical device, compressing the implantable medical device onto the carrier element such that one or more shoulders or protrusions of the implantable medical device extend internally into the bore and engage the carrier device.

22. The method according to claim 21, further including the step of radially compressing the implantable medical device such that the one or more shoulders or protrusions indent the outer wall of the carrier element.
